(19) United States
(12) Patent Application Publication
(10) Pub. No.: US 2007/0017534 A1
(43) Pub. Date: Jan. 25, 2007

(54) System and method of determining impairment of an individual

(76) Inventor: William P. Thorpe, Winchester, MA (US)

Correspondence Address: BROMBERG & SUNSTEIN LLP, 125 SUMMER STREET, BOSTON, MA, 02110-1618, US

(21) Appl. No.: 11/402,263

(22) Filed: Apr. 11, 2006

Related U.S. Application Data
(60) Provisional application No. 60/669,983, filed on Apr. 11, 2005.
(60) Provisional application No. 60/700,868, filed on Jul. 20, 2005.

(51) Int. Cl. A61B 19/00

(52) U.S. Cl. 128/898

(57) ABSTRACT

Systems and methods for determining the extent of functional physiological impairment of an individual due to the administration of an anesthetic agent. The systems and methods determine values of a set of parameters associated with saccadic eye movements of the individual at a time following such administration and compare a function of such values with normalized data.

### PRIORITY

This application claims priority from U.S. Provisional Patent Application Ser. No. 60/669,983, filed Apr. 11, 2005, and from U.S. Provisional Application Ser. No. 60/700,868, filed Jun. 20, 2005, both of which are incorporated herein by reference.

### TECHNICAL FIELD AND BACKGROUND ART

The present invention relates to determining the level of functional physiological impairment of an awake individual and, in particular, to determining the level of functional physiological impairment of an individual by utilizing measurements of saccadic eye motion.

Early studies have shown that saccadic eye movements can be used as a measure mental performance of humans. For example, an early study in 1984 demonstrated that saccadic eye movement analysis may be used as a measure of human psychomotor performance. This study, as well as follow-on studies by other investigators, was performed using one of several experimental techniques, including electro-occulography (EOG), limbus tracking, video occulography (VOG), and scleral coil implants, to examine the dynamics of saccadic movements. Specifically, saccadic duration, saccadic velocity, peak velocity and saccadic latency were recorded and analyzed. These studies, however, necessarily limited the range of allowed movements, and required voluntary movement to set targets of known position from the subject being tested.

In addition, the effects of sedative agents such as barbiturates, opiates, benzodiazepines, carbazepine, amphetamine and alcohol on the dynamics of saccadic eye movements were examined. The principal finding from these studies was that the peak velocity of saccades for a given amplitude of movement is lower in the presence of a sedating agent.

In addition to the effects of other factors such as fatigue, neurological pathology (e.g. Parkinson disease), or psychiatric conditions have recently been suggested to have an effects on saccadic movement dynamics.

These aforementioned studies investigate primarily the effects of sedation, neuropathology or psychiatric condition by analyzing change in saccadic peak velocity, or parameters associated with velocity (e.g. acceleration). However , measurements of velocity in general, typically difficult to achieve without constraining at least one of the individual or experimental environment, because measurement of velocity requires knowledge of direction as well as speed of the eye movement.

### SUMMARY OF THE INVENTION

Embodiments of the present invention are directed to a method of determining the extent of functional physiological impairment of an awake individual due to the administration of an anesthetic agent. The method of a particular embodiment includes determining values of a set of parameters associated with saccadic eye movements of the individual at a time following such administration. The method of this embodiment also includes comparing a function of such values with normalized data.

In an aspect of this embodiment, the normalized data include values derived from values of the set of parameters associated with saccadic eye movements of the individual at a time prior to the administration of the anesthetic agent.

In another aspect, the normalized data are created from measurements of saccadic eye movements taken from a relevant population of subjects.

In another aspect, the functional impairment is attributable to sedation. In this aspect, there may be provided methods to reflect the extent of sedation of the individual.

One embodiment of the present invention is directed to systems for making and analyzing saccadic eye movements. This embodiment includes an eye motion detection system for making measurements of an individual's saccadic eye movements. The system will have the capability of simultaneous binocular measurements. In addition to acquiring standard eye movement data such as saccadic speed and acceleration, the system will also measure degree of alignment between each eye. The system also will measure disconjugacy between left and right eye movements as a function of neuropathology, intoxication and/or sedation. As such, this measurement may be used as a separate component of a sedation factor. This new parameter may be self normalized and used in conjunction with other sedation factors to assess physiological functionality.

Due to the novelty of the invention design, various attributes of an individual's eye movements, unrelated to sedation, may be objectively measured. These conditions include, but are not limited to, measurement of misalignment between left and right eye positions. This misalignment may be used as a screen for pediatric strabismus and amblyopia.

The system of this embodiment may also include saccadic eye movement processing software to analyze data received from the eye motion detection system. In one aspect of this embodiment, the saccadic eye movement processing software may compare the data received from the eye motion detection system to normalized data of the saccades relevant population of subjects to determine the level of functional physiological impairment of an individual.

In some embodiments of the present invention, parameterization of saccadic eye movement, functions may allow for the assessment of the degree of functional physiological impairment of an individual due to a variety of drugs and may be used to document such individual's return to normal physiological functionality. Moreover, since a variety of influences may degrade an individual's cognitive state or physiologic functionality such as fatigue, or in general any influence which acts to decrease alertness and fitness for duty, this methodology may be used to assess these non-pharmacological influences on physiologic functionality. Because the parameters that are used to determine this physiologic functionality are independent of voluntary saccadic movement, no extraneous stimulus is needed to elicit saccades. In other words, any saccade, whether voluntary or involuntary, may be use in the analysis.

Furthermore, while individual saccades may differ significantly from individual to individual, the dynamics of the saccade and the relationship between various parameters describing the saccade does not vary significantly within a population. Therefore, by assessing a relevant population of “normal” subjects, a general baseline may be developed against which an individual's physiological functionality may be compared. Departures from baseline could be as a result of polypharmacy (ingestion of multiple drugs with combined sedative effects), administration of an anesthetic agent, intoxication by alcohol or other depressants, fatigue, neurological pathology, psychiatric pathology, or a host of other factors.

In some of embodiments of the present invention, the set of sedation factors may be developed which result from various groupings of the parameters determined during an analysis of an individuals eye movements. These sedation factors may have normal distributions that center around a mean value per measurement per subject. The sedation factors may vary with degree of alertness, or hypnotic state of the individual due to administration of anesthetic agents. The parameters that may be combined to generate these sedation factors include, but are not limited to, the square of the duration of individual eye saccades (dx)² (dx is a number resulting from a Boltzmann equation fit to the individual saccade positional data), the normalized amplitude of the eye saccade |A|, and the peak speed of each saccade as evaluated by the 1^{st} derivative with respect to time of the saccade positional data. In one embodiment, the sedation factor is the ratio of the square of the duration of an individual's eye saccades to the normalized amplitude of each saccade.

The device is also capable of merging of existing measurement technologies in order to develop new sedation factors. The device may be capable of incorporating existing EEG monitoring (e.g. BIS monitor) and specifically capture the brainwave activity resulting from saccadic burst neuron activity. The resulting saccadic movement could thus be correlated with this neuronal activity. By comparing these measurements, specific factors could be measured, such as saccadic latency, with great accuracy. This latency measurement would be reflective of any neuropathology or sedation effects on the subjects brainstem function.

In a similar fashion, other pertinent physiologic measurements could be incorporated into the system and used to determine new sedation factor or physiological functionality factors. These measurements include, but not limited to, pulse oximetry and temperature.

The device will also include lens material with the ability to change from optically clear to opaque by electronic pulse. An example of such technology is LCD lenses currently used for video gaming. Our embodiment of opacifying of lenses may include, but is not limited to LCD technology. This patching of individual eyes may be accomplished within milliseconds. This electronic ‘patching’ replicates manual patching techniques currently used by optometrists and ophthalmologist during vision screening. In the current screening mode of the device, this patching or shuttering will alternate from right to left eye while recording eye positions and movements. In addition, the device will be capable of programming a patch (total opacity) to be turned on and off for various lengths of time on either eye. This prolonged patching (hours) may be used in a therapeutic mode for the treatment of lazy eye.

### BRIEF DESCRIPTION OF THE DRAWINGS

The foregoing features of the invention will be more readily understood by reference to the following detailed description, taken with reference to the accompanying drawings, in which:

FIG. 1 is an example of one embodiment of the present invention where a forehead mounted camera is placed on a patient;

FIG. 2 is a graph of peak speeds of a number of saccades plotted against the integrated area under each saccade curve;

FIG. 3 is a plot of actual patient data showing sedation factor as a function of time pre/post op;

FIG. 4 is an example of a limbus tracker which may be used to measure saccadic or other eye movements.

### DETAILED DESCRIPTION OF SPECIFIC EMBODIMENTS

Definitions: As used herein in this description and the accompanying claims, the following terms shall have the meanings indicated, unless the context otherwise requires:

“physiological functional impairment” means the level to which objective functions of an individual are reduced. The impairment may be attributable to one or more of many external influences including, without limitation, drug ingestion, the administration of an anesthetic agent, fatigue, psychiatric disease or a neuropathology. A specific form of physiological functional impairment is sedation due to the administration of an anesthetic agent to an awake subject.

“physiological condition” of a subject means the condition of the subject in a pertinent realm that is under observation, such realm may include a state of health or disease or physical fitness.

“sedation” means the depression of an individual's awareness of the environment and reduction of his or her responsiveness to external stimulation while awake. There are two types of sedation, conscious sedation is light sedation in which the patient maintains his or her airway reflexes and ability to cooperate (subject is awake) and deep sedation is a more profound depression of the response to stimulation in which airway reflexes may not be maintained (patient is asleep).

As discussed above, embodiments of the present invention are directed to systems and methods for determining level of physiological functional impairment of an individual due to the administration of an anesthetic agent. The level of physiological functional impairment may be determined, in some embodiments, based on measurements of the individual's saccadic eye movements. Such embodiments take advantage of my discovery that various parameters associated with saccadic eye movement may be used to determine a level of impairment attributable to the administration of an anesthetic agent.

These embodiments are based on my discovery that there is a relationship between the saccadic eye movements of an individual and the level of physiological functional impairment of the individual. Indeed, various parameters associated with saccadic eye movement can be used to determine the extent of an individual's physiological functional impairment, from a baseline level of physiological functionality, that may be attributable to the administration of an anesthetic agent, so that extent of recovery of an individual from anesthesia or other outside influence may be readily assessed by determination of saccadic eye motion parameters. Furthermore, there exists a dose-response relationship between specific drugs (anesthetic agents being a specific example) and parameters associated with saccadic movement of the eye. In addition, in some embodiments, only normalized data from each subject is required for the determination of their sedation factor. In other words, in contrast to other eye monitoring devices, the device according to the present invention may not need calibration prior to use.

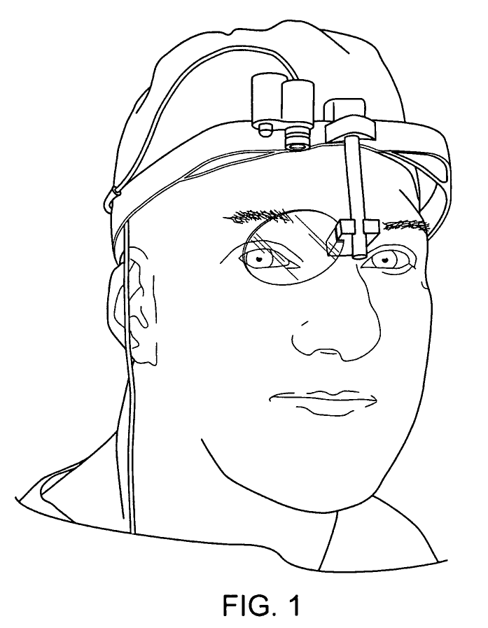

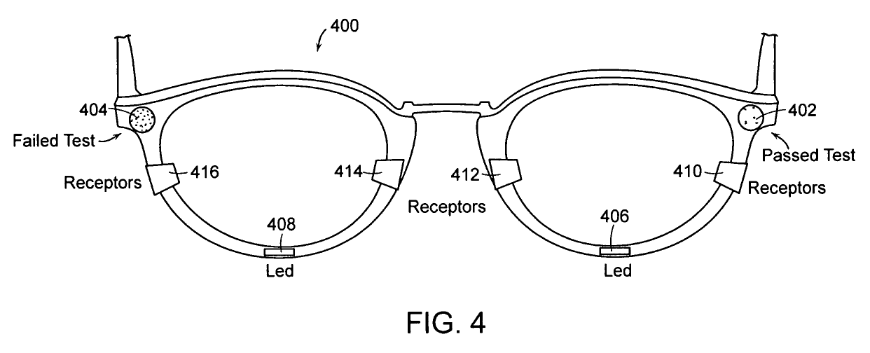

In one embodiment, the measurements of an individual's saccadic eye movements may be made by an eye motion detection system. There are many of types of eye motion detection systems that may be used for this purpose. For instance, the measurements may be made by a system that optically determines eye location using a high-speed forehead-mounted camera which acquires images of the eye that is illuminated by near infrared LED's located next to a forehead mounted camera. An example of such a camera is Model 346.1, a modified version of a head-band-mounted eye tracker supplied by ISCAN, Inc. of Burlington, Mass. This camera outputs eye position approximately 240 times per second. An example of such a camera is shown in use with a patient in the attached FIG. 1. In other embodiments, the eye motion detection system could be a limbus tracker. An example of a limbus tracker in shown in FIG. 4.

Eye motion detection system data may be interrogated to resolve saccadic motion. Particular values of the saccades are determined, as described below. In some embodiments, the values of the saccades are determined by saccadic eye movement processing software which utilizes data received from the eye motion detection system. The values of the saccades may be used as indicators of the functional physiological impairment (e.g., sedation) of a patient.

Regardless of how the measurements are made, in one embodiment, the eye motion detection system may operate at sampling rates between 240 Hz and 600 Hz. For instance, the eye motion detection system could operate at 240 Hz, 300 Hz, or 600 Hz. In an embodiment where the eye motion detection system is a limbus tracker, the eye motion detection system may operate at 600 Hz or above.

Regardless of how eye motion detection is performed, the system may include processing software (or hardware, or a combination thereof) that may analyze the data received from the eye motion tracker. Of course, a system of the present invention may include means for communicating the data to the software. For instance, the data may be communicated from the eye motion tracker to the processing software (which may be running on a CPU or a small microchip located on the tracker) by wire or in a wireless manner. If wireless, communication may be at radio frequencies (RF) and may use a communication protocol such as Bluetooth™.

In some embodiments, the following measurements may be made from the data accumulated by the eye tracker: the square of the duration of individual eye saccades (dx)² (dx is a number resulting from a Boltzmann equation fit to the individual saccade positional data), the normalized amplitude of the eye saccade |A|, and the peak speed of each saccade as evaluated by the 1^{st} derivative with respect to time of the saccade positional data. Of course, other measurements could be made without departing from the spirit of the present invention. For instance, the measurements could include a determination of the difference between the fixation point of each eye to determine if a person has, for example, lazy eye. In one embodiment, a level of physiological functional impairment may be determined by determining the ratio of the square of the duration of an individual's eye saccades to normalized amplitude of each saccade.

Eye speed may also be determined in this embodiment. In any of these embodiments, the maximum speed values may be considered to be the peaks of possible saccades. In some embodiments, anomalous high and low peaks may be filtered out by either the user or automatically.

Saccades may be recognized by interrogation of the x-y position data points just before and after the peak, as well as the elapsed time between peaks. A series of rules may be applied to the candidate saccades that account for physiological conditions expected during a saccade. Candidate saccades that do not adhere to these rules may be deemed artifact, and not considered in the data set.

In this embodiment, after the saccades have been identified, the peak normalized speeds of the saccades may then optionally be graphed with the integrated area under each saccade curve fit to the following function:
Y=A[1−e^{(-X/B)}] (1)
where Y is the peak saccadic speed, A is the maximum saccade speed, X is the area under the curve (or normalized amplitude of each saccade), and B is the saccadic amplitude 67% of saturable speed (A).

FIG. 2 is a graph of peak speeds of a number of saccades measured with the camera described above plotted against the integrated area under each saccade curve.

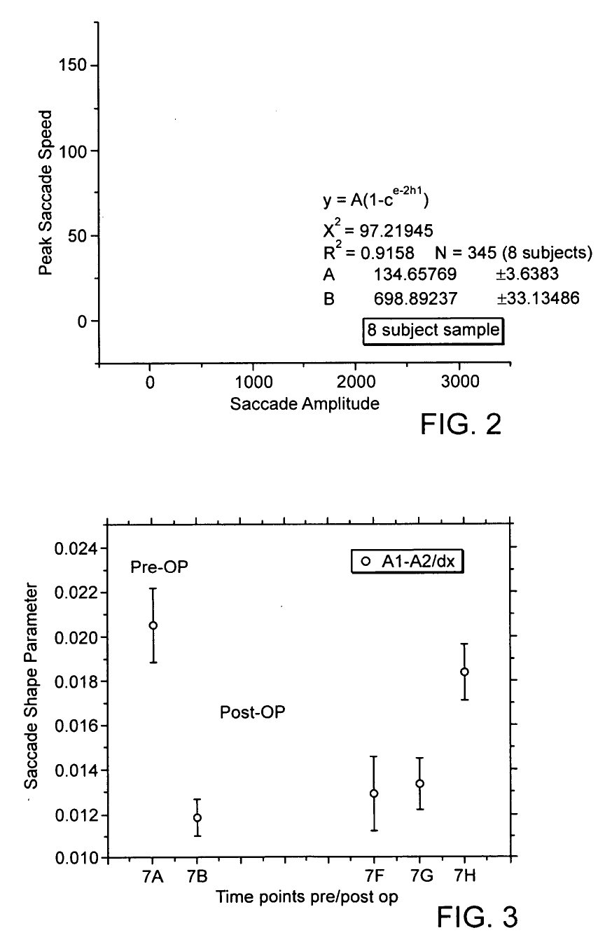

A characteristic time constant T may then be calculated for each saccade curve according to the following formula:
T=B/A (2)
In addition, the shape of each accepted saccade may be analyzed according to the following formula:
S=(A1−A2)/dx (3
where S is a shape factor, A1 and A2 are high and low amplitude of the saccade, and dx is the value of a characteristic time of duration of the saccade. By plotting the mean values of this shape factor as a function of time in the recovery room (either pre-op or various times post-op) the sedation of the patient may be evaluated by observing first a sharp decline in the Shape factor values, and eventual return to baseline (pre-op) values as the patient recovers from anesthesia. An example of this phenomenon is demonstrated in FIG. 3, which is a plot of actual patient data showing shape factor as a function of time pre/post op. The value of dx is generated from fitting the individual saccade data to a Boltzman function given by:
y=A2+(A1−A2)/(1+e^{x-x}₀^{/dx}) (4)
where the parameters A1, A2 are described above, is the normalized saccadic position and xo is the time of the saccade start, and x is each time point on the y vs. x saccadic speed curve.

Other sedation factors may include, but not be limited to combining normalized values of saccadic acceleration, saccadic duration, saccadic amplitude, binocular disconjugacy and latency. Moreover, disease specific factors may be developed based upon these parameters and could be used for diagnostic purposes. Such disease entities include ADD, ADHD, depression, bipolar disease and schizophrenia.

Embodiments of this methodology may provide normalized data of a relevant population of subjects and, thus not require comparison from the same patient before and after sedation (or other event such as alcohol consumption that might have a neurological effect). In other words, this specific embodiment may allow for the determination of a constant ‘normal’ baseline saccadic behavior for non-sedated adult individuals. In such an embodiment, the normative measurements for a population of non-sedated individuals may be used in relation to one or more post-administration measurements to determine the extent of physiological functional impairment.

Alternatively or in addition, a base line of sedated adults may also be used. In such an embodiment, the normative measurements are indicative of an extent of physiological functional impairment. Also, the data are the basis for an interrogation of individual saccades or a number of saccades taken sequentially, in terms of peak speed, shape of the speed vs. time curve and/or the time constant, as indicators of neurological state. In addition, in this embodiment, the relevant population of subjects may be selected from based on their age group, gender or physiological condition. For example, if the individual has a particular medical condition for which treatment is being provided when the individual is under sedation, then there may be a benefit in using baseline data for a population of subjects that has the particular condition. Similarly, if the individual is of an advanced age, it may be more accurate to compare the individual's saccades with those of persons similarly situated.

As discussed above, the device of the present invention may be used to make instantaneous determinations about individuals unrelated to saccades. For instance, the present invention could simultaneously determine the fixation point of a person's left and right eyes. If the difference between the two is above a certain threshold, the device could indicate that this person may suffer from ambyopia or lazy eye. For instance, and as shown in FIG. 4, the limbus tracker 400 could include “passed” 402 and “failed” 404 indicators that, in some embodiments, could instantaneously indicate whether the person does or does not have lazy eye. These indicators could be, for example, LED's or other light emitting source. In such an embodiment, the processing software may be located on a processor mounted on the tracker 400.

The tracker 400 may also include a left 406 and right 408 LED's that shine ultraviolet light (or other frequency) into the persons eyes. An example of such LED's may include model number OP200 made by TT electronics. The reflection of this light from the eye may be measured by left eye position sensors 410 and 412 and right eye position sensors 414 and 416. The fixation point may be determined by analyzing the output of the left and right eye position sensors. Examples of eye position sensors include, model OP520 made by TT electronics.

All aforementioned embodiments of the invention are intended to be merely exemplary and numerous variations and modifications will be apparent to those skilled in the art. All such variations and modifications are intended to be within the scope of the present invention as defined in the appended claims.

1. A method of determining the extent of functional physiological impairment of an individual due to the administration of an anesthetic agent comprising:
determining values of a set of parameters associated with saccadic eye movements of the individual at a time following such administration; and
comparing a function of such values with normalized data.

2. A method according to claim 1, wherein the normalized data includes values derived from values of the set of parameters associated with saccadic eye movements of the individual at a time prior to such administration.

3. A method according to claim 1, wherein the normalized data is created from measurements of saccadic eye movements taken from a relevant population of subjects.

4. A method according to claim 3, wherein the relevant population is based on one or more subjects to which an anesthetic agent has been administered.

5. A method according to claim 3, wherein the relevant population is based no one or more subjects to which anesthetic agents have not been administered.

6. A method according to claim 4, wherein the relevant population is determined based on one or more factors selected from age group, gender, and physiological condition.

7. A method according to claim 4, wherein the relevant population is determined based on one or more factors selected from age group, gender, and physiological condition.

8. A method according to claim 1, wherein the functional impairment is attributable to sedation.

9. A method according to claim 4, wherein the method reflects the extent of sedation of the individual.

10. A system for determining the extent of functional physiological impairment of an individual due to the administration of an anesthetic agent comprising:
an eye motion detection system for determining values of a set of parameters associated with saccadic eye movements of the individual at a time following such administration; and
means for comparing a function of such values with normalized data.

11. A system according to claim 10, wherein the normalized data includes values derived from values of the set of parameters associated with saccadic eye movements of the individual at a time prior to such administration.

12. A system according to claim 10, wherein the normalized data is created from measurements of saccadic eye movements taken from a relevant population of subjects.

13. A system according to claim 12, wherein the relevant population is based on one or more subjects to which an anesthetic agent has been administered.

14. A system according to claim 12, wherein the relevant population is based no one or more subjects to which anesthetic agents have not been administered.

15. A system according to claim 13, wherein the relevant population is determined based on one or more factors selected from age group, gender, and physiological condition.

16. A system according to claim 13, wherein the relevant population is determined based on one or more factors selected from age group, gender, and physiological condition.

17. A system according to claim 10, wherein the functional impairment is attributable to sedation.

18. A system according to claim 10, wherein the eye motion detection system includes a limbus tracker.

19. A system according to claim 10, wherein the eye motion detection system includes forehead mounted camera.
